(19) United States
(12) Patent Application Publication
(10) Pub. No.: US 2022/0238201 A1
(43) Pub. Date: Jul. 28, 2022

(54) TEMPORARY ALARM MODIFICATIONS IN AMBULATORY INFUSION PUMP SYSTEM

(71) Applicant: Tandem Diabetes Care, Inc., San Diego, CA (US)

(72) Inventors: Geoffrey A. Kruse, San Diego, CA (US); Virginia Lu, San Diego, CA (US); Chrstian Merz, Carlsbad, CA (US)

(21) Appl. No.: 17/587,412

(22) Filed: Jan. 28, 2022

Related U.S. Application Data
(60) Provisional application No. 63/142,803, filed on Jan. 28, 2021.
(60) Provisional application No. 63/174,836, filed on Apr. 14, 2021.

(51) Int. Cl. G16H 20/17; A61M 5/142

(52) U.S. Cl. CPC G16H 20/17; A61M 5/14248; A61M 2205/18; A61M 2205/3561; A61M 2205/3389; A61M 2205/505

(57) ABSTRACT

Disclosed herein are systems and methods for optimizing alarms, alerts, reminders and other notifications in an ambulatory infusion pump system.

### RELATED APPLICATION

This application claims the benefit of U.S. Provisional Application No. 63/142,803 filed Jan. 28, 2021, and to U.S. Provisional Application No. 63/174,836 filed Apr. 14, 2021, each of which is hereby fully incorporated herein by reference.

### FIELD OF THE INVENTION

The present invention relates generally to ambulatory infusion pumps and, more particularly, to alerts, alarms and reminders issued by ambulatory infusion pump systems.

### BACKGROUND OF THE INVENTION

There are a wide variety of medical treatments that include the administration of a therapeutic fluid in precise, known amounts at predetermined intervals. Devices and methods exist that are directed to the delivery of such fluids, which may be liquids or gases, are known in the art.

One category of such fluid delivery devices includes insulin injecting pumps developed for administering insulin to patients afflicted with type 1, or in some cases, type 2 diabetes. Some insulin injecting pumps are configured as portable or ambulatory infusion devices that can provide continuous subcutaneous insulin injection and/or infusion therapy as an alternative to multiple daily insulin injections via syringe or injector pen. Such ambulatory infusion pumps may be worn by the user, may use replaceable medicament cartridges, and may deliver other medicaments alone, or in combination with insulin. Such medicaments include glucagon, pramlintide, and the like. Examples of such pumps and various features associated therewith include those disclosed in U.S. Patent Publication Nos. 2013/0324928 and 2013/0053816 and U.S. Pat. Nos. 8,287,495; 8,573,027; 8,986,253; and 9,381,297, each of which is incorporated herein by reference in its entirety. Ambulatory infusion pump systems can monitor a number of conditions relating to the pump and treatment with the pump. Such systems can therefore be configured to automatically provide various alerts, alarms and reminders to a user when a monitored condition requires user action or the user should otherwise be informed of the condition. However, not all alerts, alarms and reminders are critical and there may be times where a user would not want to be disturbed by a notification, particularly for less urgent or critical notifications. In addition, a user may not always be capable of properly responding to a given alert based on the user's location, access to supplies, etc.

### SUMMARY OF THE INVENTION

Disclosed herein are systems and methods for optimizing alarms, alerts, reminders and other notifications in an ambulatory infusion pump system to avoid disruptions during sleep periods and/or other periods when a user would not want to be disturbed. A Do Not Disturb (DND) algorithm can execute a DND schedule that matches sleep activity for a user and/or other periods where a user does not want to be disturbed. The DND algorithm can predict any notifications that would occur during the DND schedule and review such notifications. Depending on the type of notification and its urgency, the DND algorithm can decide to cancel the notification, defer the notification until after the DND schedule or preemptively issue the notification prior to implementing the DND schedule.

Also disclosed herein are systems and methods for optimizing alarms, alerts, reminders and other notifications in an ambulatory infusion pump system. Often such notifications can occur overnight and continue to reoccur disturbing the user's sleep or when the user is in a location where the user cannot properly address the notifications. Embodiments herein therefore provide notifications to user's that are more convenient and more tailored to the user's schedule.

In an embodiments, an ambulatory infusion pump system includes a pump mechanism configured to deliver medicament to a user, a user interface and at least one processor configured to cause the pump mechanism to deliver medicament to the user and to provide notifications to the user on the user interface. The processor can be configured to selectively activate a do not disturb feature that includes determining any notifications that would occur while the do not disturb feature is active. Any non-critical notifications that would occur while the do not disturb feature is active can then be cancelled. Any critical notifications that can be deferred until after the do not disturb feature is deactivated can be deferred. Any critical notifications that cannot be deferred until after the do not disturb feature is deactivated prior can be preemptively provided prior to implementing the do not disturb feature.

In an embodiment, an ambulatory infusion pump system can include a refillable reservoir configured to contain a medicament, a pump mechanism configured to deliver medicament from the reservoir to a user, a user interface and at least one processor configured to cause the pump mechanism to deliver medicament to the user and display information on the user interface. The processor can be configured to determine that medicament is being added into the refillable reservoir and to determine an amount of medicament in the reservoir after the medicament is added. The processor can estimate an amount of time that the amount of medicament will provide therapy to the user before the refillable reservoir will need additional medicament. A message can be provided to the user on the user interface relating to the amount of time that the amount of medicament will provide therapy to the user before the refillable reservoir will need additional medicament.

In an embodiment, an ambulatory infusion pump system includes a pump mechanism configured to deliver medicament to a user, a user interface and at least one processor configured to cause the pump mechanism to deliver medicament to the user and display information on the user interface. The processor can be configured to receive user input defining a location for location-based therapy notifications and determine a location of the user relative to the location for location-based therapy notifications. One or more therapy notifications can selectively be provided to the user based on the location of the user relative to the location for location-based therapy notifications.

### BRIEF DESCRIPTION OF THE DRAWINGS

The invention may be more completely understood in consideration of the following detailed description of various embodiments of the invention in connection with the accompanying drawings, in which:

FIG. 1 is an embodiment of an ambulatory infusion pump for use with embodiments of the disclosure.

FIG. 2 is a block diagram of the ambulatory infusion pump of FIG. 1.

FIGS. 3A-3B are an alternate embodiment of an ambulatory infusion pump for use with embodiments of the disclosure.

FIG. 4 depicts an embodiment of an infusion pump system according to the disclosure.

FIGS. 5A-5B depict remote control devices for an infusion pump system according to the disclosure.

FIG. 6 depicts a flowchart of methods steps for a Do Not Disturb algorithm according to the disclosure.

FIG. 7 depicts a cartridge estimator feature for an infusion pump system according to an embodiment.

FIG. 8 depicts a flowchart of method steps for setting geographic therapy notifications according to an embodiment.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION OF THE INVENTION

The following detailed description should be read with reference to the drawings in which similar elements in different drawings are numbered the same. The drawings, which are not necessarily to scale, depict illustrative embodiments and are not intended to limit the scope of the invention.

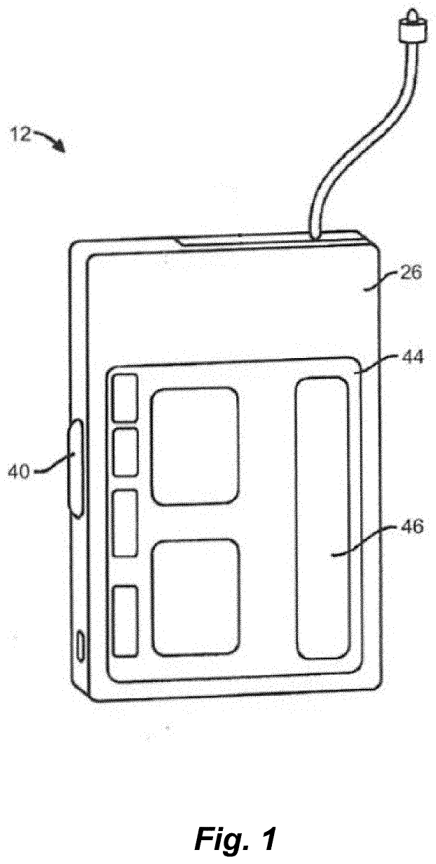

FIG. 1 depicts an example infusion pump that can be used in conjunction with one or more embodiments of the ambulatory infusion pump system of the present disclosure. Pump 12 includes a pumping or delivery mechanism and reservoir for delivering insulin or other medicaments to a patient and an output/display 44. The output/display 44 may include an interactive and/or touch sensitive screen 46 having an input device such as, for example, a touch screen comprising a capacitive screen or a resistive screen. The pump 12 may additionally or instead include one or more of a keyboard, a microphone or other input devices known in the art for data entry, some or all of which may be separate from the display. The pump 12 may also include a capability to operatively couple to one or more other display devices such as a remote display (e.g., a dedicated remote display or a CGM display), a remote control device, or a consumer electronic device (e.g., laptop computer, personal computer, tablet computer, smartphone, electronic watch, electronic health or fitness monitor, or personal digital assistant). Further details regarding such pump devices can be found in U.S. Pat. No. 8,287,495, previously incorporated by reference above. It is to be appreciated that pump 12 may be optionally configured to deliver one or more additional or other medicaments to a patient.

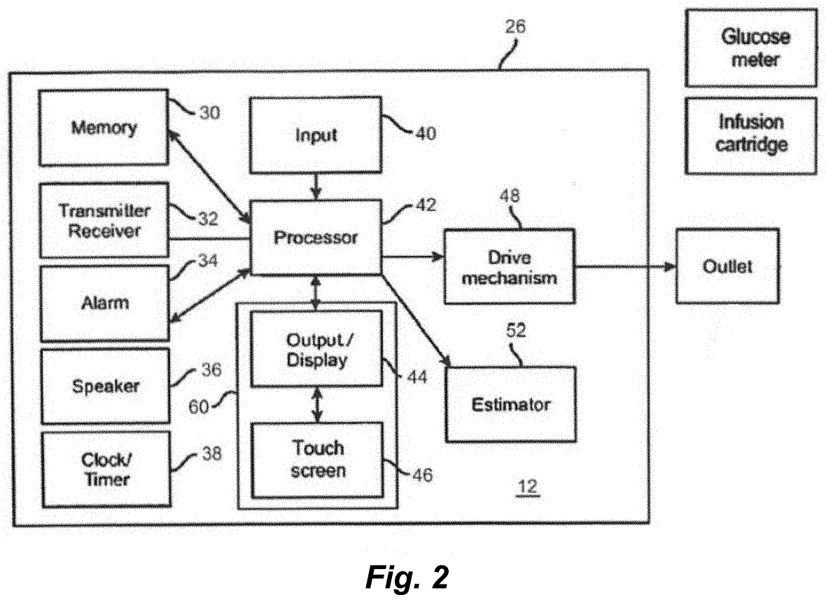

FIG. 2 illustrates a block diagram of some of the features that may be included within the housing 26 of pump 12. The pump 12 can include a processor 42 that controls the overall functions of the pump. The pump 12 may also include, e.g., a memory device 30, a transmitter/receiver 32, an alarm 34, a speaker 36, a clock/timer 38, an input device 40, a user interface suitable for accepting input and commands from a user such as a caregiver or patient, a drive mechanism 48, an estimator device 52 and a microphone (not pictured). One embodiment of a user interface is a graphical user interface (GUI) 60 having a touch sensitive screen 46 with input capability. In some embodiments, the processor 42 may communicate with one or more other processors within the pump 12 and/or one or more processors of other devices through the transmitter/receiver 32 such as a remote device (e.g., CGM device), a remote control device, or a consumer electronic device. In some embodiments, the communication is effectuated wirelessly, by way of example only, via a near field communication (NFC) radio frequency (RF) transmitter or a transmitter operating according to a “Wi-Fi” or Bluetooth® protocol, Bluetooth® low energy protocol or the like. The processor 42 may also include programming to receive signals and/or other data from an input device, such as, by way of example, a pressure sensor, a temperature sensor, an accelerometer, a GPS receiver, or the like.

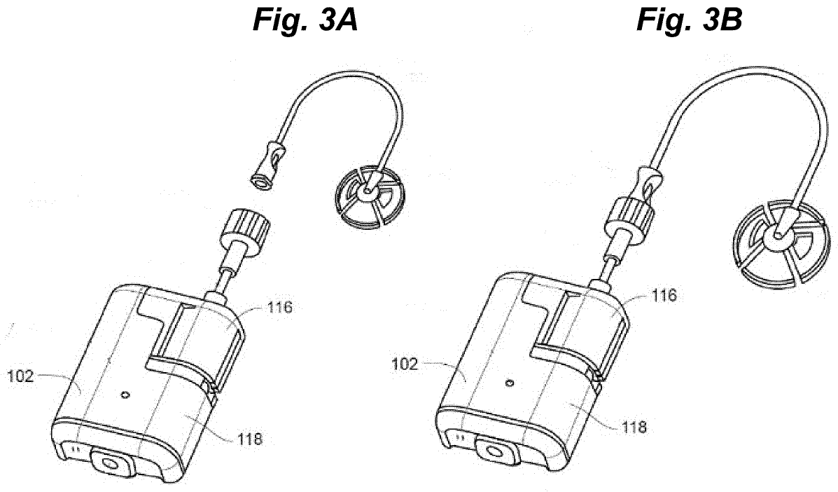

FIGS. 3A-3B depicts another infusion pump that can be used in conjunction with one or more embodiments of the ambulatory infusion pump system of the present disclosure. Pump 102 includes a pump drive unit 118 and a medicament cartridge 116. Pump 102 also includes a processor that controls some or all of the operations of the pump. The processor may communicate with one or more processors within the pump 102 and/or one or more processors of other devices. The processor may also include programming to receive signals and/or other data from an input device, such as, by way of example, a pressure sensor, a temperature sensor, or the like. In some embodiments, pump 102 receives commands from a separate device for control of some or all of the operations of the pump. Such separate device can include, for example, a dedicated remote control device or a consumer electronic device such as a smartphone having a processor executing an application configured to enable the device to transmit operating commands to the processor of pump 102. In some embodiments, processor can also transmit information to one or more separate devices, such as information pertaining to device parameters, alarms, reminders, pump status, etc. Such separate device can include any remote display, remote control device, or a consumer electronic device as described above. Pump 102 can also incorporate any or all of the features described with respect to pump 12 in FIG. 2. In some embodiments, the communication is effectuated wirelessly. Further details regarding such pumps can be found in U.S. Pat. No. 10,279,106 and U.S. Patent Publication Nos. 2016/0339172 and 2017/0049957, each of which is hereby incorporated herein by reference in its entirety.

In some embodiments, all elements of an infusion pump system such as, e.g., the user interface, processor(s), pump mechanism, etc., reside in a single device, such as an infusion pump. In other embodiments, an infusion pump system may be a distributed system in which portions of the functionality such as, e.g., the user interface, speaker, processor, dosing algorithm, etc. may reside in separate devices such as in the infusion pump, dedicated remote control and/or other mobile device such as a mobile phone, or central computer system such as a cloud computing system.

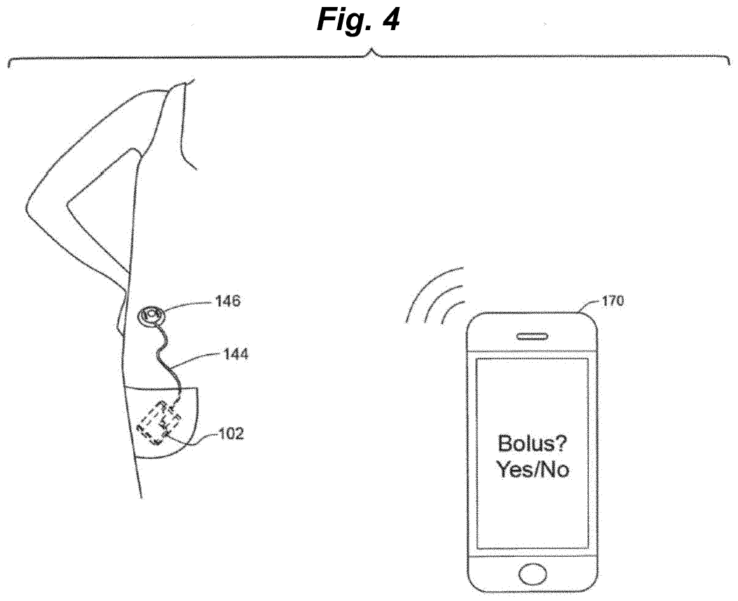

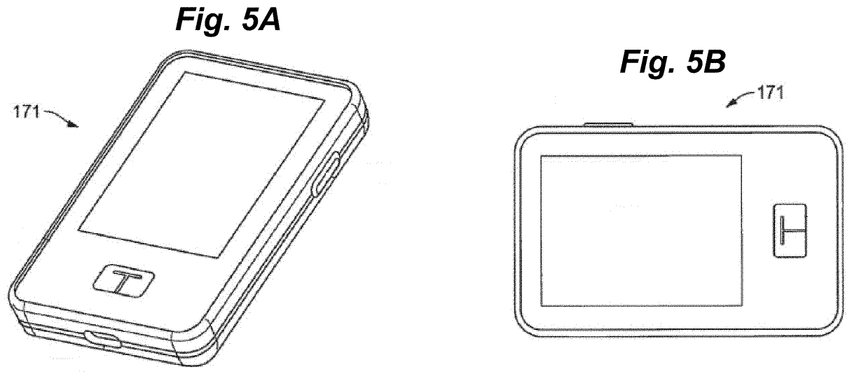

Referring to FIGS. 4-5B, one or more remote control devices 170, 171 can be used to communicate with the processor of pump 12 or pump 102 to control delivery of medicament and transfer data with pump via a wired or a wireless electromagnetic signal, such as via, e.g., a near field communication (NFC) radio frequency (RF) modality or other RF modalities such as Bluetooth®, Bluetooth® low energy, mobile or Wi-Fi communication protocols, for example, according to embodiments of the present disclosure. Such a remote control can include, for example, a mobile communication device 170, such as a smartphone (as depicted in FIG. 4) executing a software application for control of the pump, a dedicated remote controller 171 (as depicted in FIGS. 5A-5B), a wearable electronic watch or electronic health or fitness monitor or personal digital assistant (PDA), etc., or a tablet, laptop or personal computer. Such communications between (and among) the one or more remote control devices 170, 171 and pump may be one-way or two-way for, e.g., effective transfer of data among the devices and the pump, control of pump operations, updating software on the devices and/or pump, and allowing pump-related data to be viewed on the devices and/or pump.

Embodiments of the present invention include components capable of and methods using wired and wireless transmission and receipt of signals for exchange of information and commands between and among any of the components as described herein, including, e.g., between a pump and a smartphone; among a pump, a CGM and a smartphone; between a dedicated remote controller and a pump; among a dedicated remote controller, a CGM and a pump; among a dedicated remote controller, a BGM and a pump, and other combinations as would be contemplated by those of skill in the art.

Ambulatory infusion pump systems as described herein can monitor a number of conditions relating to the pump and treatment with the pump including, for example, an amount of medicament in the pump, a battery level of the pump, infusion set usage, therapy delivered with the pump, glucose levels of the user obtained from a continuous glucose monitor (CGM), CGM sensor and transmitter expiration, and others. Such systems can therefore be configured to automatically provide various alerts, alarms and reminders to a user when a monitored condition requires user action, or the user should otherwise be informed of the condition. Because there may be circumstances in which a user does not want to be disturbed by such notifications, embodiments disclosed herein can include do not disturb features that can cancel, delay, or preemptively provide various notifications to the user in order to not disturb a user during a given time period. Such notifications can be given, for example, on a user interface of the pump, a remote control device, a smartphone configured to control the pump, a continuous glucose monitor or any other device in an ambulatory infusion pump system.

In particular, a number of alarms, alerts and other notifications can occur overnight that can disrupt the sleep of a patient and the patient's family. Embodiments herein can therefore employ algorithms that can pre-emptively address such notifications by either delivering the notification before the user goes to sleep or cancelling or delaying the notification until after the user wakes up. In some embodiments, a user can program a sleep schedule into the system. In other embodiments, the system can use action-oriented feedback to detect when a user is asleep and/or to set a sleep schedule for a user. The sleep schedule can further be manually turned on/off by the user or automatically entered by the system according to a programmed sleep schedule.

In an embodiment, a Do Not Disturb or “Disturb Me Less” feature can temporarily deactivate non-critical notifications over a predetermined period of time and preemptively issue certain notifications prior to the sleep time. For example, the system can set a Do Not Disturb (DND) schedule that matches sleep activity either programmed into or detected by the system. During the scheduled DND period, the system can turn off and not issue non-critical alarms and alerts and can expedite or defer other alerts for before or after the sleep time. In embodiments, once a sleep schedule is programmed or established, the DND schedule can exactly match the sleep schedule, or can also extend for a predetermined time before and/or after the sleep schedule to better enable the user to fall asleep and wake up without receiving non-critical notifications.

Notifications can be reviewed and modified based on the timing and urgency of each notification. Any critical notifications that would need to be addressed during the DND schedule, such as, for example, a low battery notification for a battery that would not last through the night to the end of the DND schedule, could be preemptively given prior to the DND schedule. In embodiments, such preemptive notifications can be given a predetermined period of time, e.g, 30 minutes, 1 hour, etc. prior to the DND period as selected by a user or preprogrammed into the system. If a notification can be deferred until after the DND schedule, the notification can be delayed and given at the expiration of the DND period. For example, if a CGM sensor will be 12 hours from expiration during the sleep period that would otherwise trigger a Sensor Expiring Soon alert during the DND schedule, because the sensor will not actually expire during the sleep time the alert is not critical and the algorithm can defer the notification to instead occur at the end of the DND period (with an updated time until expiration). Some alerts have escalations that are given as a condition becomes more critical or imminent. For example, the system can issue a Low Battery alert at a first low battery level and a later Very Low Battery alert at a second, lower battery level. If multiple such alerts are deferred during the DND schedule, only the most critical alert (e.g., Very Low Battery) need be given at the expiration of the DND schedule and any previous alerts can be cancelled.

In some embodiments, the DND algorithm can continue executing even if the user interacts with the device during the DND schedule. For example, if a user wakes up during the night to have a snack and programs a bolus, the system can continue to defer the alerts until after the DND schedule rather than giving any deferred alerts when the user interacts with the device.

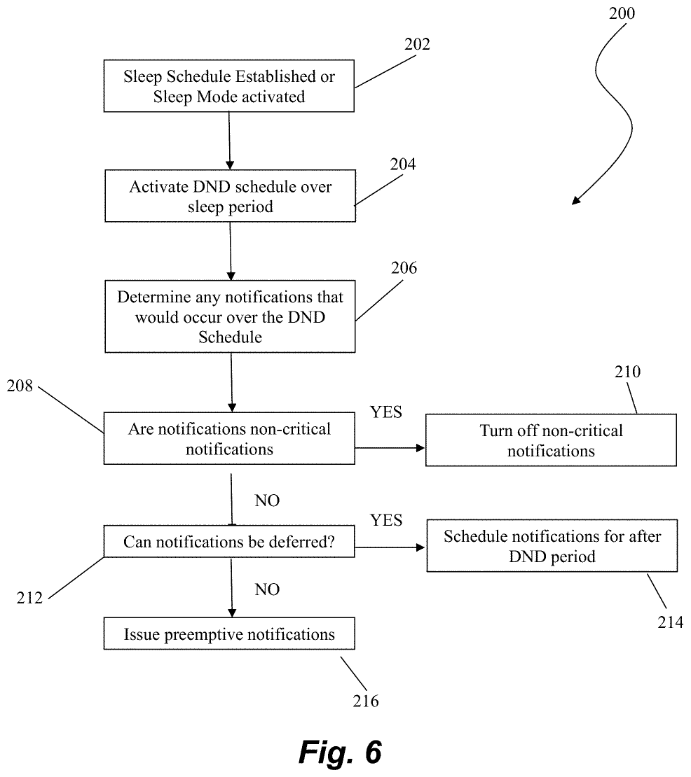

FIG. 6 depicts a flowchart of steps taken by a Do Not Disturb algorithm 200 in implementing a DND schedule according to an embodiment. At step 202, a sleep schedule is established or a sleep mode is otherwise activated as set forth above. The DND schedule is then implemented over the sleep period defined by the sleep schedule at step 204. Upon activating the DND schedule, at step 206 the algorithm determines if any alerts, alarms or other notifications would occur during the sleep period. At step 208, the algorithm determines if any of the notifications that would occur during the sleep period are non-critical notifications. Any notifications that are non-critical can by turned off and skipped at step 210. Notifications that cannot be skipped can then be reviewed at step 212 to determine if the notifications can be deferred until after the DND period. Such notifications can then be scheduled to be given after the DND period at step 214. Notifications that cannot be skipped or deferred can be given preemptively prior to the DND period at step 216. If any unexpected critical alerts occur while the DND schedule is operating, the DND feature can be temporarily deactivated to allow the system to issue any such critical alerts while the user is sleeping.

One example of an alert that can be addressed with the DND feature described herein is a Low Battery Alert that can alert the user of a low battery level at one or more thresholds. The low battery level can be based on a percentage of battery power remaining, or could be user-specific with the algorithm estimating a remaining time of battery life based on the user's actual use of the device, such as how often the user interacts with the device, how many operations are programmed to be carried out by the device, whether the device is regularly communicating with a CGM or other device, etc. If the battery would reach one or more of the thresholds for a Low Battery Alert, but the battery would still have power after the programmed sleep time, the alert can be deferred and given at the expiration of the DND schedule. If the battery will or is likely to run out of power during the DND period, the Low Battery Alert can be preemptively issued prior to (e.g., one hour before) the sleep activity. Such an alert can in embodiments include an estimation as to when during the sleep period the battery would die or can generally inform the user that the battery would run out of power while the user is sleeping. If the user does not respond to the alert by sufficiently charging the battery and it is determined during the DND period that the battery will run out of power during the period, the DND schedule can be temporarily deactivated in order to issue a critical Low Battery Alert.

Another example of an alert that can be modified by a DND algorithm as described herein is a Low Insulin Alert that monitors an amount of insulin (or other medicament) remaining in the pump reservoir and can provide alerts at various low insulin thresholds. The algorithm can calculate, based on the user's basal rate(s) during the sleep period and/or historical trends if there is sufficient insulin to last through the DND period. If there is sufficient insulin for the sleep time, any Low Insulin Alerts that would be given during the DND schedule can be deferred until after the sleep activity concludes (with only the most critical alert given and any prior alerts cancelled). If the amount of insulin in the reservoir is not likely to last through the sleep time, the alert can be given preemptively to give the user the opportunity to fill the reservoir and avoid a sleep disruption. As with other critical alerts, if the user does not sufficiently fill the reservoir and the reservoir will run out of insulin during the sleep time, the DND schedule can be temporarily deactivated to issue a critical Low Insulin Alert as needed.

Infusion pump systems can track how long an infusion set that delivers insulin from the pump into the body has been in use and provide reminders for changing to new infusion sets after a predetermined period of time (e.g., every 24 to 72 hours). If the infusion set is going to expire during the sleep time, the DND algorithm can preemptively remind the user to change the infusion set before the DND schedule is implemented. This can decrease the risk of an occlusion in the infusion set while the user is asleep that could lead to high glucose levels or diabetic ketoacidosis. In some embodiments, if the user does not respond to the preemptive reminder by changing the infusion set, the reminder that would occur during the DND schedule could then be deferred and issued after the DND period. For example, the user may have noticed the preemptive reminder and checked the infusion set and found it to be working properly and therefore did not change the infusion set for that reason, rather than simply ignoring the reminder.

Alerts for changing CGM sensors and CGM transmitters are typically given based on a predetermined time that transmitters and sensors have been in use and can also be modified by a DND algorithm as disclosed herein. If a sensor or transmitter is set to expire during the sleep time, the user can be preemptively alerted to change the sensor or transmitter prior to institution of the DND schedule. If a sensor or transmitter will last through the DND period, an alert indicating that the hardware will expire soon can be deferred and issued upon conclusion of the DND schedule. If a sensor or transmitter expires unexpectedly during the night, or if the user did not change the sensor or transmitter in response to a preemptive notification, the user can be alerted during the DND period.

Some insulin pumps include an auto-off feature that automatically turns off the pump and stops insulin delivery if the user has not interacted with the pump over a predetermined period of time (e.g., 12 hours). The DND algorithm can determine if the auto-off feature would be activated during the DND schedule (e.g., if the user hasn't interacted with the pump for a number of hours before the sleep time) and preemptively alert the user that the auto-off feature would be activated while the user is sleeping. In some embodiments, if the user does not respond to the alert and the pump would turn off during the DND period, the DND schedule could be temporarily suspended in order to issue the alert to the user that the pump is going to turn off Although the DND algorithm is primarily described herein with respect to a sleep schedule, it should be understood that the DND algorithm can alternatively or additionally be based off of various other schedules or activities. For example, the DND algorithm could be applied during meetings or appointments of the user and other events that can be obtained from an interactive calendar of a user such as can be found on a smartphone or other computing device of the user. Based on the particular alert and the timing of the activity, the algorithm can determine whether to preemptively issue a notification, defer a notification or cancel a notification as described herein. In some embodiments, the algorithm can utilize GPS or other location data to determine whether to preemptively issue alerts. For example, if it is determined that a user is leaving the user's home, an alert can be preemptively issued if, e.g., a low battery, low insulin in the reservoir or other condition that may be able to be more easily addressed from home is expected to occur in the near future when the user may be away from home. Alerts can also be modified based on other locations, such as deferring or cancelling alerts while a user is located in a church, for example.

As noted above, one notification that can be provided in infusion pump system is a low insulin (or other medicament) alert that can provide an alert to the user to refill the insulin cartridge when the amount of insulin remaining in the cartridge reaches a predetermined level, such as, e.g. 10-40 units. Often, this alert is triggered at nighttime and may reoccur through the night when the user may not be able to refill the cartridge and/or otherwise waking the user up and disturbing the user's sleep. Embodiments disclosed herein can therefore include a cartridge load estimator to aid in preemptively providing such notifications.

In an embodiment, an infusion pump system can include a cartridge load estimator that can utilize a preferred cartridge fill time and/or day to estimate when the cartridge will need to be filled and provide an alert to the user. For example, when the cartridge is empty and the user fills the cartridge with insulin (or other medicament), the user can indicate when he or she would like to fill the cartridge again, such as, for example, in 3 days at 5:00 pm. User fill preferences can be entered when the user fills the cartridge and/or previously stored in memory. The system can estimate the amount of insulin needed in the cartridge to last until the selected time and inform the user of how much insulin will be needed to meet the user's preference. The estimated amount of insulin needed can be calculated based on the user's profile settings and past therapy decisions, including automatic delivery adjustments, manually delivered meal and correction boluses, and temporary basal rates.

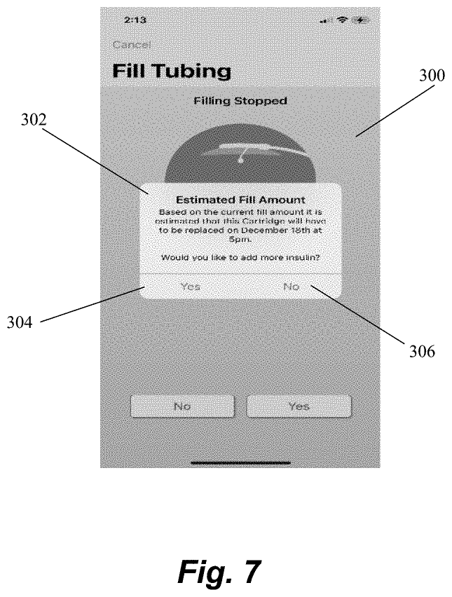

The cartridge load estimator can alternatively or additionally inform the user how long the amount of insulin added to the cartridge will last when the cartridge is filled and ask whether the user would like to add more insulin. Referring to FIG. 7, a message 302 provided by a cartridge load estimator feature of an infusion pump system on a user interface 300 of, e.g., an infusion pump, a dedicated remote control device, a smartphone configured to control an infusion pump, etc. during a cartridge filling procedure is depicted. When the cartridge is filled, the cartridge estimator can calculate when the cartridge will again be empty as set forth above and message 302 can inform the user of the approximate time and date when the cartridge will need to be filled again. The message can further ask if the user would like to add more insulin if the displayed time and date is not preferred by the user and provide a YES 304 icon that the user can select in order to fill the cartridge further and alter the calculated fill date and time. If the displayed date and time are acceptable to the user, the user can select the NO 306 icon to proceed with the cartridge filling and loading process.

In another embodiment, the user can alternatively or additionally set a customized alert setting a time and/or day at which the cartridge estimator can inform the user if the cartridge needs to be filled. For example, a user may program the feature to inform the user if the cartridge will need to be filled prior to the next morning (e.g., 8:00 am) by a certain time each night (e.g., 8:00 pm). If the cartridge will need to be filled prior to 8:00 am on an upcoming morning, the alert informing the user that the cartridge will need to be filled can be provided at 8:00 pm the night before. If the cartridge will not need to be filled, either the system can inform the user that the cartridge will not be filled or no alert is given, according to user preference. The user can alternatively or additionally set a day of the week or date for such an alert. For example, the system can inform the user if the cartridge is going to need to be filled by a certain day and/or time (e.g., Sunday at 5:00 pm) prior to a certain time (e.g., Friday at 5:00 pm).

Although specifically described with respect to a cartridge load estimator that estimates an amount of time that insulin will remain in the cartridge to preemptively provide an alert regarding filling of the cartridge, it should be understood that the above concepts can be applied to any other alert that can occur overnight or another inconvenient time that could be given at a more convenient time for the user. Such alerts include, for example, a low battery alert, CGM session ends soon, CGM transmitter expires soon, etc. by having the system estimate when, e.g., the battery will die, CGM transmitter expire, etc. In another embodiment, therapy notifications such as to fill a cartridge can be based on a location area of the user. A user may be able to enable therapy notifications based on a virtual geographic boundary, i.e., geofence. One or more enabled notifications can then automatically be provided when it is detected that the user is within and/or leaves the geofence. Location of the user can be obtained from a device associated with the user, such as the user's pump, remote control and/or smartphone by any available means, such as, for example, GPS, Wi-Fi, RFID, cellular data, etc. A location area or vicinity can be defined by one or more GPS locations and/or a defined radius around one or more GPS locations.

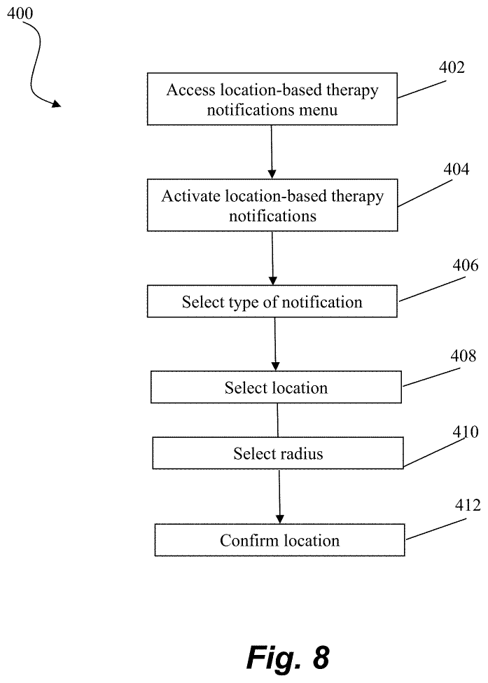

Referring now to FIG. 8, a flowchart of method steps for setting geographic therapy notifications 400 is depicted. At step 402, the user accesses a location-based therapy notifications menu. The location-based therapy notifications menu can be accessed on a user interface of an infusion pump, a dedicated remote control, a smartphone, a tablet, laptop or desktop computer, etc. At step 404 the user can choose to activate location-based therapy notifications in the menu by, for example, selecting a “Notify Me” or other menu item.

The user can select a type of notification to enable for location-based therapy notifications at step 406. Notifications can include, for example, low insulin alert, low battery alert, CGM session ends soon, CGM transmitter expires soon, etc. A location around which the location-based notifications are based can be selected at step 408. Such locations can be selected based on, e.g., an address, a name of a place, a GPS or other coordinate location, etc. At step 410, the user can select a radius around the entered location to define the geofence for the notification. In one embodiment, the user can select whether to have the notification triggered within a predefined small, medium, or large radius about the location. At step 412 the user can confirm the programmed notification. In some embodiments, once a location-based therapy notification is initially programmed, the user can have the option to selectively enable and disable the notification. The user may also be able to select to have the notification provided when the user arrives within the geofence, leaves the geofence or both. Other options can include a selection of a time period for the notifications. For example, the notification can be given only if the need for the notification would arise within a certain period of time of the user being inside or outside of the geofence. More than one type of notification may be able to be selected for a given geofence.

For example, a user may leave home to go to work and only find out that his/her insulin cartridge does not have enough insulin to last the full workday once the user arrives at work. This may be inconvenient as the user may not have insulin available at the workplace to refill the cartridge. With the location-based notifications described herein, the user could establish a geofence based on the user's home address that provides an alert to the user regarding the amount of insulin remaining in the cartridge and/or how long the insulin in the cartridge will last. In some embodiments, the user can automatically be notified how long the insulin in the cartridge will last any time the user leaves the geofence at the user's home. Alternatively, the user can program a time period such as, e.g., 8 hours, and be notified only if the insulin in the cartridge will not last through the programmed time period. Similarly, a user could establish a geofence based at the user's doctor's office and a reminder to upload data from the user's pump could automatically be given any time the user enters the geofence around the doctor's office. In a further embodiment, data could be automatically uploaded from the pump when the user enters the doctor's office. Other automatic pump actions based on a user leaving or arriving at a geofence are also contemplated.

In embodiments, an ambulatory infusion pump system includes a pump mechanism configured to deliver medicament to a user, a user interface and at least one processor configured to cause the pump mechanism to deliver medicament to the user and to provide notifications to the user on the user interface. The processor can be configured to selectively activate a do not disturb feature that includes determining any notifications that would occur while the do not disturb feature is active. Any non-critical notifications that would occur while the do not disturb feature is active can then be cancelled. Any critical notifications that can be deferred until after the do not disturb feature is deactivated can be deferred. Any critical notifications that cannot be deferred until after the do not disturb feature is deactivated prior can be preemptively provided prior to implementing the do not disturb feature.

In some embodiments, the at least one processor can be configured to automatically activate the do not disturb feature.

In some embodiments, the at least one processor can be configured to automatically activate the do not disturb feature based on a preprogrammed sleep schedule for the user. In some embodiments, the at least one processor can be configured to preemptively provide any critical notifications that cannot be deferred a predetermined time before the do not disturb feature is automatically activated.

In some embodiments, the at least one processor can be configured to receive user input through the user interface activating the do not disturb feature.

In some embodiments, the at least one processor can be configured to deactivate the do not disturb feature after a predetermined period of time.

In some embodiments, the at least one processor can be further configured to temporarily deactivate the do not disturb feature to issue any unanticipated critical notifications that occur while the do not disturb feature is active.

In embodiments, an ambulatory infusion pump system can include a refillable reservoir configured to contain a medicament, a pump mechanism configured to deliver medicament from the reservoir to a user, a user interface and at least one processor configured to cause the pump mechanism to deliver medicament to the user and display information on the user interface. The processor can be configured to determine that medicament is being added into the refillable reservoir and to determine an amount of medicament in the reservoir after the medicament is added. The processor can estimate an amount of time that the amount of medicament will provide therapy to the user before the refillable reservoir will need additional medicament. A message can be provided to the user on the user interface relating to the amount of time that the amount of medicament will provide therapy to the user before the refillable reservoir will need additional medicament.

In some embodiments, the message can inform the user when the reservoir will need the additional medicament.

In some embodiments, the message can ask the user whether the user would like to add more medicament.

In some embodiments, the message can inform the user of an approximate date and time when the additional medicament will need to be added to the reservoir.

In some embodiments, the at least on processor can further be configured to store a user preference for a time of day to be notified if the refillable reservoir will need additional medicament prior to a predefined subsequent time of day, and the at least one processor can be configured to provide the message to the user at the time of day if additional medicament will be needed prior to the predefined subsequent time of day.

In some embodiments, the at least one processor can further be configured to store a user preference for a preferred time when the user would like to refill the cartridge and the message can inform the user how much medicament will need to be in the reservoir for the medicament to be sufficient for therapy until the preferred time.

In some embodiments, the at least one processor can be configured to estimate the amount of time that the amount of medicament will provide therapy to the user before the refillable reservoir will need additional medicament based on medicament delivery history for the user.

In embodiments, an ambulatory infusion pump system includes a pump mechanism configured to deliver medicament to a user, a user interface and at least one processor configured to cause the pump mechanism to deliver medicament to the user and display information on the user interface. The processor can be configured to receive user input defining a location for location-based therapy notifications and determine a location of the user relative to the location for location-based therapy notifications. One or more therapy notifications can selectively be provided to the user based on the location of the user relative to the location for location-based therapy notifications.

In some embodiments, the at least one processor can be configured to provide the one or more therapy notifications if it is determined that the user is leaving the location for location-based therapy notifications.

In some embodiments, the at least one processor can be configured to provide the one or more therapy notifications if it is determined that the user is entering the location for location-based therapy notifications.

In some embodiments, the at least one processor can be configured to determine the location of the user based on a GPS location of a user device.

In some embodiments, the least one processor can be further configured to receive a radius around the location for location-based therapy notifications.

In some embodiments, the at least one processor can be further configured to receive a selection of one or more types of notification from a plurality of types of notifications to be provided as location-based notifications.

Although embodiments described herein may be discussed in the context of the controlled delivery of insulin, delivery of other medicaments, singly or in combination with one another or with insulin, including, for example, glucagon, pramlintide, etc., as well as other applications are also contemplated. Device and method embodiments discussed herein may be used for pain medication, chemotherapy, iron chelation, immunoglobulin treatment, dextrose or saline IV delivery, treatment of various conditions including, e.g., pulmonary hypertension, or any other suitable indication or application. Non-medical applications are also contemplated.

With regard to the above detailed description, like reference numerals used therein may refer to like elements that may have the same or similar dimensions, materials, and configurations. While particular forms of embodiments have been illustrated and described, it will be apparent that various modifications can be made without departing from the spirit and scope of the embodiments herein. Accordingly, it is not intended that the invention be limited by the forgoing detailed description.

The entirety of each patent, patent application, publication, and document referenced herein is hereby incorporated by reference. Citation of the above patents, patent applications, publications and documents is not an admission that any of the foregoing is pertinent prior art, nor does it constitute any admission as to the contents or date of these documents.

Also incorporated herein by reference in their entirety are commonly owned U.S. Pat. Nos. 6,999,854; 8,133,197; 8,287,495; 8,408,421 8,448,824; 8,573,027; 8,650,937; 8,986,523; 9,173,998; 9,180,242; 9,180,243; 9,238,100; 9,242,043; 9,335,910; 9,381,271; 9,421,329; 9,486,171; 9,486,571; 9,492,608; 9,503,526; 9,555,186; 9,565,718; 9,603,995; 9,669,160; 9,715,327; 9,737,656; 9,750,871; 9,867,937; 9,867,953; 9,940,441; 9,993,595; 10,016,561; 10,201,656; 10,279,105; 10,279,106; 10,279,107; 10,357,603; 10,357,606; 10,492,141; 10/541,987; 10,569,016; 10,736,037; 10,888,655; 10,994,077; and 11,116,901. commonly owned U.S. Patent Publication Nos. 2009/0287180; 2012/0123230; 2013/0053816; 2014/0276423; 2014/0276569; 2014/0276570; 2018/0071454; 2019/0240398; 2019/0307952; 2020/0114076; 2020/0206420; 2020/0261649; 2020/0306445; 2020/0329433; 2020/0368430; 2020/0372995; 2021/0001044;

2021/0113766; 2021/0154405; and 2021/0353857 and commonly owned U.S. patent application Ser. Nos. 17/368,968; 17/459,129; and Ser. No. 17/517,885.

Modifications may be made to the foregoing embodiments without departing from the basic aspects of the technology. Although the technology may have been described in substantial detail with reference to one or more specific embodiments, changes may be made to the embodiments specifically disclosed in this application, yet these modifications and improvements are within the scope and spirit of the technology. The technology illustratively described herein may suitably be practiced in the absence of any element(s) not specifically disclosed herein. The terms and expressions which have been employed are used as terms of description and not of limitation and use of such terms and expressions do not exclude any equivalents of the features shown and described or portions thereof and various modifications are possible within the scope of the technology claimed. Although the present technology has been specifically disclosed by representative embodiments and optional features, modification and variation of the concepts herein disclosed may be made, and such modifications and variations may be considered within the scope of this technology.

1. An ambulatory infusion pump system, comprising:
a pump mechanism configured to deliver medicament to a user;
a user interface; and
at least one processor configured to cause the pump mechanism to deliver medicament to the user and to provide notifications to the user on the user interface, the at least one processor further configured to selectively activate a do not disturb feature including the at least one processor:
determining any notifications that would occur while the do not disturb feature is active;
cancelling non-critical notifications if any non-critical notifications would occur while the do not disturb feature is active;
deferring critical notifications that can be deferred until after the do not disturb feature is deactivated if any such critical notifications would occur while the do not disturb feature is active; and
preemptively providing prior to implementing the do not disturb feature critical notifications that cannot be deferred until after the do not disturb feature is deactivated if any such critical notifications would occur while the do not disturb feature is active.

2. The ambulatory infusion pump system of claim 1, wherein the at least one processor is configured to automatically activate the do not disturb feature.

3. The ambulatory infusion pump system of claim 2, wherein the at least one processor is configured to automatically activate the do not disturb feature based on a preprogrammed sleep schedule for the user.

4. The ambulatory infusion pump system of claim 2, wherein the at least one processor is configured to preemptively provide any critical notifications that cannot be deferred a predetermined time before the do not disturb feature is automatically activated.

5. The ambulatory infusion pump of claim 1, wherein the at least one processor is configured to receive user input through the user interface activating the do not disturb feature.

6. The ambulatory infusion pump system of claim 1, wherein the at least one processor is configured to deactivate the do not disturb feature after a predetermined period of time.

7. The ambulatory infusion pump system of claim 1, wherein the at least one processor is further configured to temporarily deactivate the do not disturb feature to issue any unanticipated critical notifications that occur while the do not disturb feature is active.

8. An ambulatory infusion pump system, comprising:
a refillable reservoir configured to contain a medicament;
a pump mechanism configured to deliver medicament from the reservoir to a user;
a user interface; and
at least one processor configured to cause the pump mechanism to deliver medicament to the user and display information on the user interface, the at least one processor further configured to:
determine that medicament is being added into the refillable reservoir;
determine an amount of medicament in the reservoir after the medicament is added;
estimate an amount of time that the amount of medicament will provide therapy to the user before the refillable reservoir will need additional medicament; and
providing a message to the user on the user interface relating to the amount of time that the amount of medicament will provide therapy to the user before the refillable reservoir will need additional medicament.

9. The system of claim 8, wherein the message informs the user when the reservoir will need the additional medicament.

10. The system of claim 9, where the message asks the user whether the user would like to add more medicament.

11. The system of claim 9, wherein the message informs the user of an approximate date and time when the additional medicament will need to be added to the reservoir.

12. The system of claim 8, wherein the at least on processor is further configured to store a user preference for a time of day to be notified if the refillable reservoir will need additional medicament prior to a predefined subsequent time of day, and the at least one processor is configured to provide the message to the user at the time of day if additional medicament will be needed prior to the predefined subsequent time of day.

13. The system of claim 8, wherein the at least one processor is further configured to store a user preference for a preferred time when the user would like to refill the cartridge and wherein the message can inform the user how much medicament will need to be in the reservoir for the medicament to be sufficient for therapy until the preferred time.

14. The system of claim 8, wherein the at least one processor is configured to estimate the amount of time that the amount of medicament will provide therapy to the user before the refillable reservoir will need additional medicament based on medicament delivery history for the user.

15. An ambulatory infusion pump system, comprising:
a pump mechanism configured to deliver medicament to a user;
a user interface; and
at least one processor configured to cause the pump mechanism to deliver medicament to the user and display information on the user interface, the at least one processor further configured to:
receive user input defining a location area for location-based therapy notifications;
determine a location of the user relative to the location area for location-based therapy notifications; and
selectively provide one or more therapy notifications to the user based on the location of the user relative to the location area for location-based therapy notifications.

16. The ambulatory infusion pump system of claim 15, where the at least one processor is configured to provide the one or more therapy notifications if it is determined that the user is leaving the location area for location-based therapy notifications.

17. The ambulatory infusion pump system of claim 15, where the at least one processor is configured to provide the one or more therapy notifications if it is determined that the user is entering the location area for location-based therapy notifications.

18. The ambulatory infusion pump system of claim 15, wherein the at least one processor is configured to determine the location of the user based on a GPS location of a user device.

19. The ambulatory infusion pump system of claim 15, wherein the least one processor is further configured to receive a radius around the location area for location-based therapy notifications.

20. The ambulatory infusion pump system of claim 15, wherein the at least one processor is further configured to receive a selection of one or more types of notification from a plurality of types of notifications to be provided as location-based notifications.
